(19) United States
(12) Patent Application Publication
(10) Pub. No.: US 2009/0281077 A1
(43) Pub. Date: Nov. 12, 2009

(54) Cyclic sulfones useful as mitochondrial sodium-calcium exchangers

(76) Inventors: Samuel Hintermann, Basel (CH); Michele Chiesi, Blotzheim (FR)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/440,440

(22) PCT Filed: Sep. 7, 2007
(86) PCT No.: PCT/EP07/59394

§ 371 Date: Mar. 6, 2009

(30) Foreign Application Priority Data
Sep. 8, 2006 (EP) 06120389.9

(51) Int. Cl. A61K 31/554; C07D 281/02; A61P 25/00; A61P 9/00

(52) U.S. Cl. 514/211.05; 540/490

(57) ABSTRACT

The invention relates to novel heterocyclic compounds of the formula

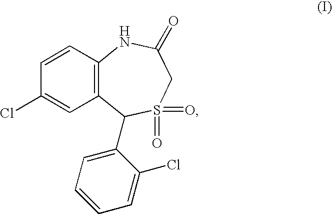

in free base form or in acid addition salt form, to their preparation, to their use as medicaments and to medicaments comprising them.

The present invention relates to novel heterocyclic compounds, to their preparation, to their use as medicaments and to medicaments comprising them.

More particularly the invention relates to a compound of the formula

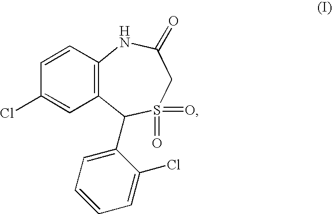

in free base form or in acid addition salt form.

E. g. on account of the asymmetrical carbon atom present in a compound of the formula I, a compound of the formula I may exist in pure optically active form or in the form of a mixture of optical isomers, e. g. in the form of a racemic mixture. All of such pure optical isomers and all of their mixtures, including the racemic mixtures, are part of the present invention.

A compound of the formula I may exist in free base form or in acid addition salt form. All of such free compounds and salts are part of the present invention.

A compound of the formula I may exist in tautomeric form. All of such tautomers are part of the present invention.

In especially preferred embodiments, the invention relates to one or more than one of the compounds of the formula I mentioned in the Examples, in free base form or in acid addition salt form.

In a further aspect, the invention relates to a process for the preparation of a compound of the formula I, in free base form or in acid addition salt form, comprising the steps of reacting a compound of the formula

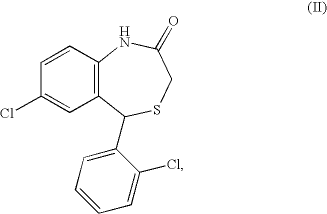

in free base form or in acid addition salt form, with an oxidizing agent, optionally followed by the cleavage of any protecting group(s) optionally present, and of recovering a so obtainable compound of the formula I in free base form or in acid addition salt form.

The process steps can be effected according to conventional methods, for example as described in the Examples.

As oxidizing agent in the oxidizing step can be used, for example, ozone, a dioxirane derivative, such as dimethyldioxirane, an oxidizing pyridinium salt, such as pyridinium chlorochromate, a peroxide, such as H₂O₂ or tert.-butylhydroperoxide, an inorganic peracid or a salt thereof, such as KHSO₅, a composition comprising an inorganic peracid or a salt thereof, such as OXONE®, or an organic peracid, such as peracetic acid or meta-chloroperbenzoic acid.

The oxidizing step can be carried out in the presence of a solvent, which is preferably inert under the reaction conditions employed.

The cleavage of a protecting group may be carried out in accordance with known procedures.

The working-up of a reaction mixture and the purification of a compound of the formula I thus obtainable may be carried out in accordance with known procedures.

An acid addition salt of a compound of the formula I may be produced from the corresponding free base in known manner, and vice-versa.

The starting materials of the formula II are known or may be prepared according to conventional procedures starting from known compounds.

A compound of the formula I can also be prepared by further conventional processes, which processes are further aspects of the invention.

The compounds of the formula I and their pharmaceutically acceptable acid addition salts, hereinafter referred to as “agents of the invention”, exhibit valuable pharmacological properties when tested in vitro and in animals, and are therefore useful as medicaments.

The agents of the invention are inhibitors of the mitochondrial sodium-calcium exchanger (mNCE), which is an important component of the mitochondrial Ca-homeostasis in excitable tissues. Therefore, the agents of the invention can be used for the treatment and/or prevention of disorders or diseases influenced by the malfunction of the mitochondrial Ca-handling capacity.

The agents of the invention are, therefore, useful, e. g., for the treatment and/or prevention of neurological, vascular or metabolic disorders or diseases, such as neurodegenerative diseases, e. g. Parkinson's disease (PD), Alzheimer's disease (AD), Huntington's disease, multiple sclerosis (MS), Down's syndrome, memory impairment, cognitive impairment, dementia, neuronal degeneration, brain inflammation, myasthenia gravis, nerve trauma, brain trauma, progressive supranuclear palsy, amyotrophic lateral sclerosis (ALS), amyotrophic lateral sclerosis—(ALS)-like syndrome, aging, Leber's hereditary optic neuropathy (LHON) syndrome, Leigh's syndrome, mitochondrial encephalomyopathy, lactic acidosis and stroke-like episodes (MELAS) syndrome, familial bilateral striatal necrosis (FBSN) syndrome, growth retardation, aminoaciduria, lactic acidosis and early death (GRACILE) syndrome, myoclonic epilepsy with ragged-red fibers (MERRF) syndrome, neuropathy, ataxia and retinitis pigmentosa (NARP) syndrome, progressive external ophtalmoplegia (PEO) syndrome, Kearns-Sayre (KS) syndrome, sudden infant death (SID) syndrome, dominant optic atrophy, mtDNA depletion (MD) syndrome, Barth's syndrome, mitochondrial neurogastrointestinal encephalomyopathy, Mohr-Tranebjaerg's syndrome, Friedreich's ataxia, Wilson's disease, pathological conditions following ischemia-reperfusion damage (such as cardiac ischemia or stroke), pathological conditions following epileptic seizures, Niemann-Pick type C disease, diabetes (such as type 1 diabetes, type 2 diabetes or juvenile onset diabetes) or atherosclerosis.

For the above-mentioned indications, the appropriate dosage of an agent of the invention will of course vary depending upon, for example, the compound employed, the host, the mode of administration or the nature and severity of the condition being treated and/or prevented. However, in general, satisfactory results in animals are indicated to be obtained at a daily dosage of an agent of the invention of from about 0.1 to about 100, preferably from about 1 to about 50, mg/kg of animal body weight. In larger mammals, for example humans, an indicated daily dosage is in the range of from about 10 to about 2000, preferably from about 10 to about 200, mg of an agent of the invention conveniently administered, for example, in divided doses up to four times a day or in sustained release form.

The agent of the invention may be administered by any conventional route, in particular enterally, preferably orally, for example in the form of tablets or capsules, or parenterally, for example in the form of injectable solutions or suspensions.

In accordance with the foregoing, the present invention also provides an agent of the invention for use as a medicament, e. g. for the treatment and/or prevention of disorders or diseases influenced by the malfunction of the mitochondrial Ca-handling capacity.

The present invention furthermore provides a pharmaceutical composition comprising an agent of the invention in association with at least one pharmaceutical carrier or diluent. Such compositions may be manufactured in conventional manner. Unit dosage forms contain, for example, from about 1 to about 1000, preferably from about 1 to about 500, mg of an agent of the invention.

An agent of the invention can be administered alone or as a combination with at least one other pharmaceutical agent, which combination is effective in the treatment and/or prevention of conditions mentioned above.

The pharmaceutical combination may be in the form of a unit dosage form, whereby each unit dosage will comprise a predetermined amount of the active components, in admixture with suitable pharmaceutical carriers or diluents. Alternatively, the combination may be in the form of a package containing the active components separately, e. g. a pack or dispenser-device adapted for the concomitant or separate administration of the active agents, wherein these agents are separately arranged.

Moreover the present invention provides the use of an agent of the invention for the manufacture of a medicament for the treatment and/or prevention of disorders or diseases influenced by the malfunction of the mitochondrial Ca-handling capacity.

In still a further aspect, the present invention provides a method for the treatment and/or prevention of disorders or diseases influenced by the malfunction of the mitochondrial Ca-handling capacity in a subject in need of such treatment and/or prevention, which comprises administering to such subject a therapeutically effective amount of an agent of the invention.

The following Examples illustrate the invention, but do not limit it.

### EXAMPLES

### Abbreviations

BSA bovine serum albumin
DMSO dimethylsulfoxide
EDTA ethylene diamine tetraacetic acid
h hour(s)
Hepes 2-[4-(2-hydroxyethyl)-1 -piperazino]-ethanesulfonic acid
¹H-NMR proton nuclear magnetic resonance spectrometry
min minute(s)
MS mass spectrometry
rt room temperature
sec second(s)
Tris α,α,α-tris-(hydroxymethyl)-methylamine hydrochloride

### Example 1

### 2-Chloro-9-(2-chlorophenyl)-8,8-dioxo-5,7,8,9-tetrahydro-8lambda*6*-thia-5-aza-benzocyclohepten-6-one

To a stirred solution of 2-chloro-9-(2-chlorophenyl)-5,9-dihydro-8-thia-5-aza-benzocyclohepten-6-one (200 mg, 0.62 mmol) in dichloromethane (5 ml) meta-chloroperbenzoic acid (319 mg, 0.93 mmol) is added at rt. After 45 min, the mixture is taken up in dichloromethane, washed with saturated Na₂S₂O₃-solution and with saturated Na₂CO₃-solution, dried over Na₂SO₄ and concentrated. The residue is recrystallized from methanol/dichloroethane (5 ml/1 ml) yielding the title compound in the form of a white solid.

¹H-NMR (400 MHz, DMSO-D₆): 4.27 (d, J=13.7 Hz, 1H), 4.46 (d, J =13.3 Hz, 1H), 5.96 (s, 1H), 7.08 (d, J=2.4 Hz, 1H), 7.36 (d, J=8.6 Hz, 1H), 7.58-7.72 (m, 4H), 8.27 (dd, J=7.8 Hz and 1.6 Hz, 1H). MS: [M+NH₄]⁺=372.9, 374.9.

### Example 2

### Isolation of Rat Brain Mitochondria

The method is adapted from the method of Rosenthal et al. [J. Cereb. Blood Flow Metab., 7, 752-758 (1987)].

### Solutions

MSH+: 225 mM mannitol, 75 mM sucrose, 5 mM Hepes, 0.5 mM EDTA, 1 mg/ml of BSA (essentially free fatty acid free); final pH=7.3.

MSH−: Equal to MSH+, but without EDTA.

Nagarse solution: 5 mg of nagarse (bacterial protease type XXIV from Sigma, St. Louis, USA, catalogue # P-8038) dissolved in 1 ml of MSH+.

Digitonin solution: 10% W/V in DMSO.

### Procedure

The entire procedure is performed on ice. Remove the whole brain from the rat (1 brain=2 g). Add the tissue to cold MSH+in a beaker on ice. Cut the tissue into small pieces using scissors and wash the tissue 2 times with MSH+. Transfer the tissue into a 20 ml Dounce homogenizer and set level to about 9 ml with MSH+. Add 1 ml of freshly made Nagarse solution. Homogenize (6 strokes with a loose and 6 strokes with a tight pestle). Add MSH+ to make about 30 ml/brain and centrifuge at 2000 g/3 min. Keep the supernatant. Resuspend the pellet in about 30 ml of MSH+ and centrifuge again at 2000 g/3 min. Pool the supernatants and centrifuge at 12000 g /8 min. Suspend the pellet (composed mainly of mitochondria and synaptosomes) in 20 ml of MSH+/brain. Add 20 μl of digitonin solution and incubate for 2 min on ice. Centrifuge at 12000 g/10 min. Resuspend the pellet in 10 ml of MSH− and centrifuge at 12000 g/10 min. Resuspend the final pellet in 1.5 ml of MSH−/brain. The protein concentration is determined using the Pierce BCA assay (Pierce, Rockford Ill., USA). A typical yield is 10-15 mg of mitochondrial protein/rat brain (i. e. the final mitochondrial suspension is about 8 mg/ml). The mitochondria are kept on ice and are used within 2-3 h.

### Example 3

### Measurement of the Na-Ca Exchanger of Rat Brain Mitochondria

The method is basically as described by Chiesi et al. [Biochem. Pharmacol., 37, 4399-4403 (1988)] and adapted to a microtiter plate format.

### Incubation Medium

120 mM KCl, 20 mM Tris (pH=7.4), 5 μM rotenone, 10 mM K-succinate, 1 μM Oregon Green (from Molecular Probes).

### Procedure

In a typical experiment 96-well microtiter plates (flat bottom) are used. The experiment is performed at rt. Distribute in a 96-well plate 5 μl/well of 2 μM ruthenium red [in 120 mM KCl and 20 mM Tris (pH=7.4)] and the compounds (in DMSO) to be analyzed (1 μl/well). The controls receive the same amount of vehicle. Prepare the mitochondrial suspension (30 μl of freshly prepared brain mitochondria are diluted in 1 ml of incubation medium). After 4 min, when the energized mitochondria have accumulated all endogenous and contaminating Ca, 90 μl/well of mitochondrial suspension are distributed to the wells of the microtiter plate, which is then placed in a fluorimeter equipped with a syringe. The syringe is filled with 200 mM NaCl, and the fluorimeter is programmed to deliver 10 μl/well (final concentration: 20 mM) from the syringe and to make 20 measurements (once every 3 sec). Measure the Ca-release induced by the addition of Na by monitoring the Oregon Green fluorescence (485 nm and 538 nm monochromators for excitation and emission, respectively).

### Data Analysis

The evaluation of the exponentially decaying Ca-efflux curves is done by fitting and calculating the initial decay rates. To assess the efficacy of a compound, the concentration dependency curves of Ca-efflux rates are fitted using the Levenberg/Marqwardt equation to obtain IC₅₀ values.

The agents of the invention show IC₅₀ values below 20 μM in this test.

Specifically, the agent of the invention described in Example 1 shows an IC₅₀ value of 3.8 μM in this test.

1. A compound of the formula 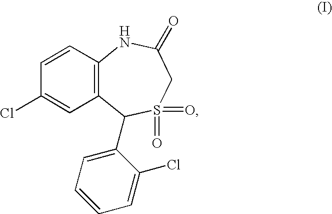
in free base form or in acid addition salt form.

2. A process for the preparation of a compound of the formula I, 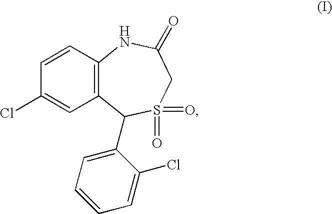
in free base form or in acid addition salt form, comprising the steps of:
reacting a compound of the formula 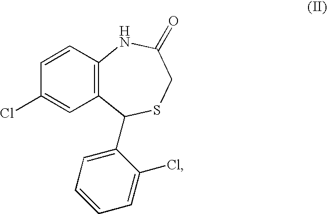
in free base form or in acid addition salt form, with an oxidizing agent, optionally followed by the cleavage of any protecting group(s) optionally present, and
recovering the compound of the formula I in free base form or in acid addition salt form.

3-4. (canceled)

5. A pharmaceutical composition, comprising:
a the compound according to claim 1, in free base form or in pharmaceutically acceptable acid addition salt form, as active ingredient and
a pharmaceutical carrier or diluent

6-7. (canceled)

8. A method for the treatment and/or prevention of disorders and diseases influenced by the malfunction of the mitochondrial Ca-handling capacity in a subject in need of such treatment and/or prevention, comprising:
administering to such subject a therapeutically effective amount of the compound according to claim 1, in free base form or in pharmaceutically acceptable acid addition salt form.

9. A combinations comprising:
a therapeutically effective amount of the compound according to claim 1, in free base form or in pharmaceutically acceptable acid addition salt form, and a second drug substance, for simultaneous or sequential administration.
